(19) United States
(12) Patent Application Publication
(10) Pub. No.: US 2006/0269494 A1
(43) Pub. Date: Nov. 30, 2006

(54) New Ubiquitin-Proteasome Regulating Compounds and Their Application in Cosmetic and Pharmaceutical Formulations

(76) Inventor: Shyam K Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(21) Appl. No.: 10/908,816

(22) Filed: May 27, 2005

(51) Int. Cl. A61K 31/7048; A61K 31/7034; A61K 8/63; A61K 8/35; A61K 31/366; A61K 31/353

(52) U.S. Cl. 424/70.1; 424/94.63; 514/27; 514/456; 514/733; 514/453; 514/25; 514/680

(57) ABSTRACT

This invention provides certain ubiquitinylation modulating molecular chaperone compounds and their use in the prevention and/or treatment of ailments associated with the dysfunction of ubiquitin-proteasome pathway, including inflammation, wound healing, skin aging, body enzyme dysfunction, neurodegenerative disorders, and cellular apoptosis.

The body constantly produces proteins and degrades proteins that are no longer needed or are defective. The production and destruction of proteins, called protein turnover, is a constant, ongoing process that is crucial for tissue renewal. A well-nourished person synthesizes nearly one pound of protein per day. Proteins that are broken down balance this protein gain. The process of protein breakdown, called proteolysis, is essential to cell survival. Numerous proteolytic systems exist in mammalian cells, the most important of which are the lysosomes, the ubiquitin-proteasome pathway, and enzymes called calpains. Lysosomes are small cell components that contain specific enzymes (proteases), which break down proteins. In the ubiquitin-proteasome pathway, proteins that are to be degraded are first marked by the addition of ubiquitin molecules and then broken down by large protein complexes called proteasomes. Calpains are proteases that are involved in several physiological processes, including the breakdown of proteins that give cells their shape and stability. The ubiquitin-proteasome system is now considered the major system involved in intracellular protein degradation. Two major components of this system are (1) three enzymes that add a small protein called ubiquitin onto substrate proteins destined for degradation, and (2) the proteasome, a rather large cellular particle composed of several smaller protein subunits, which executes the actual proteolysis. By degrading short-lived regulatory proteins, the ubiquitin-proteasome system controls basic cellular processes such as cell division, cell signaling, and gene regulation. The system also removes misfolded, damaged proteins, and in certain immune cells it breaks down foreign proteins into pieces called antigenic peptides, which can then be transported to the cell surface to induce an immune response [Ulrich, Current Topics in Microbiology and Immunology, vol 268, 137-174 (2002)].

The present invention relates to compounds that are selective regulators of Ubiquitin-proteasome pathway, to cosmetic and pharmaceutical compositions containing them, and to their use in the prevention and/or treatment of ailments associated with the dysfunction of ubiquitin-proteasome pathway, including inflammation, wound healing, skin aging, body enzyme dysfunction, neurodegenerative disorders, and cellular apoptosis.

Ubiquitin is a small protein that occurs in most eukaryotic cells. Its main function is to mark other intracellular proteins for destruction, known as proteolysis. Several ubiquitin molecules attach to the condemned protein (polyubiquitination), and polyubiquitinylated protein then moves to a proteasome, a barrel-shaped structure where the proteolysis occurs. Ubiquitin can also mark transmembrane proteins (for example, receptors) for removal from the membrane.

Ubiquitin consists of 76 amino acids with two sequentially linked glycine moieties at the carboxyl terminal and has a molecular mass of about 8500 amu (FIG. 1). It is highly conserved among eukaryotic species: Human and yeast ubiquitin share 96% amino acid sequence identity.

FIG. 1.

The process of marking a protein with ubiquitin consists of a series of steps; (1) Activation of ubiquitin—the carboxyl group of the terminal glycine of ubiquitin binds to the sulfhydryl group —SH of an ubiquitin-activating enzyme E1. The sulfhydryl group is a cysteine residue on the E1 protein. This step requires an ATP molecule as an energy source and results in the formation of a thioester bond between ubiquitin and E1; (2) Transfer of ubiquitin from E1 to the ubiquitin-conjugating enzyme E2 via trans (thio) esterification; (3) Then, the final transfer of ubiquitin to the target protein can occur either directly from E2 (this is primarily used when ubiquitin is transferred to another ubiquitin already in place, creating a branched ubiquitin chain) or via an E3 enzyme, which binds specifically to both E2 and the target protein. The target protein is usually a damaged or non-functional protein that is recognized by a destruction-targeting sequence. Ubiquitins then bind to a lysine residue in the target protein via the transformation of thioester bond into an iso-peptide bond (FIG. 2), eventually forming a tail of at least four ubiquitin molecules. The resulting ubiquitin-linked protein, called ubiquitin-protein conjugate, then can be recognized and degraded by the proteasome into peptides. This is the typical way to mark specific proteins for proteolysis. A functional proteasome (also called 26S proteasome) is composed of a smaller barrel-shaped core and two “caps” that are attached to the each end of the core. The proteasome core consists of four stacked rings containing two types of subunits, all facing into a central cavity. These subunits together have at least five distinct proteinase activities that cleave proteins at different sites. The “caps” at each end of proteasome perform a regulatory function. Each cap is composed of multiple subunits with numerous functions. These subunits recognize the ubiquitinylated protein, cut off the ubiquitin chains from this protein, thereby “unfolding” the protein, and open the channel inside the proteasome core so that th2 protein can enter the channel for degradation; and (4) Finally, the marked protein is digested in the 26S-proteasome into small peptides, amino acids (usually 6-7 amino acid subunits). Although the ubiquitins also enter the proteasome, they are not degraded (despite their protein structure) and may be used again.

FIG. 2.

Proteasomes are large multi-subunit protease complexes, localized in the nucleus and cytosol, which selectively degrade intracellular proteins. Proteasomes play a major role in the degradation of many proteins that are involved in cell cycling, proliferation, and apoptosis. Ubiquitin-proteasome pathway is illustrated in FIG. 3.

FIG. 3.

Intracellular proteolysis is the most recently discovered regulatory system of cellular physiology. Everything from cell division, development, and differentiation to cellular senescence has a proteolytic component. There is no simpler way to stop a physiological process than to destroy one of the components of a pathway in a controlled fashion. The discovery of the role of ubiquitin in the proteolytic pathway earned Aaron Ciechanover, Avram Hershko and Irwin Rose the 2004 Nobel Prize in Chemistry. Several books have become available that further reveal the importance of ubiquitins in human biology and human disease control, some of which are included herein for reference only: Ubiquitin and the Chemistry of Life, Mayer et al., John Wiley, 2005; Ubiquitin, Rechsteiner et al, Plenum Press, 1988; The Ubiquitin System, Schlesinger et al., Cold Spring Harbour Lab, 1988; Ubiquitins and the Biology of the Cell, Peters et al., Plenum Press, 2001; Self-Perpetuating Structural States in Biology, Disease, and Genetics, Proceedings of the National Academy of Sciences (2002).

A wide variety of neurodegenerative disorders are associated with the accumulation of ubiquitinylated proteins (if they are not further degraded by Proteasomes) in neuronal inclusions, and also with signs of inflammation. In these disorders, the ubiquitinylated protein aggregates, which will be seen as a foreign body by immune system, may themselves trigger the expression of inflammatory mediators, such as cyclooxygenase 2 (COX-2). Impairment of ubiquitin-proteasome pathway may contribute to this neurodegenerative and inflammatory processes. Products of COX-2, such as prostaglandin J2, can, in turn, increase the levels of ubiquitinylated proteins and also cause COX-2 up-regulation, creating a self-destructive feedback mechanism [Zongmin Li et al., International Journal of Biochemistry and Cell Biology, vol. 35, 547-552 (2003)], as shown in FIG. 4.

FIG. 4.

The gene, whose disruption causes Angelman syndrome, UBE3A, encodes an ubiquitin ligase (E3) enzyme termed E6-AP.

The ubiquitin pathway is thought to be the method of cellular egress for a number of retroviruses, including HIV and Ebola, but the exact mechanism by which this occurs has yet to be deduced. Ubiquitin has also been implicated as key components in other biochemical processes. Ubiquitinylation of the Gag structural protein of Rous Sarcoma virus has been linked to the targeting of Gag to the cell membrane of the host cell where it can assemble into spherical particles and bud from the cell surface. Production of HIV particles has also been associated with ubiquitinylation and may constitute an important cellular pathway for producing infectious particles. Thus, the ubiquitin pathway may be an important target for treatment of HIV positive patients.

The disruption of the Ubiquitin-proteasome pathway can result from damaging events, such as aging-induced decrease in proteasome function [Carrard et al., International Journal of Biochemistry and Cell Biology, vol. 34, 1461 (2002)], oxidative stress [Shringarpure et al., Free Radical Biology Medicine, vol. 32, 1084-1089 (2002)], and production of neurotoxic molecules from mutations. A dysfunctional ubiquitin-proteasome pathway may then cause proteins that are normally turned over by this pathway to aggregate and form inclusions. One of the mechanisms by which the abnormal accumulation of ubiquitinylated proteins may mediate neurodegradation is by triggering an inflammatory response. Inflammation is a natural defense against diverse insults, intended to remove damaging agents and to inhibit their detrimental effects. Treatment of neurons with proteasome inhibitors, oxidative stressors, or cyclopentenone prostaglandin J2 elicits accumulation of ubiquitinylated proteins and cytotoxicity in a concentration-dependent manner. These agents also increased the neuronal levels of COX-2 and prostaglandin E2. COX-2 is the pro-inflammatory and inducible form of cyclooxygenases, which are enzymes that catalyze the rate-limiting step in the biosynthesis of prostaglandins, prostacyclins, or thromboxane A2 from their precursor arachidonic acid. Cyclooxygenases are bifunctional hemoproteins that catalyze the cyclooxygenation of arachidonic acid to PGG2 followed by the hydroperoxidation of PGG2 to PGH2. Specific enzymes, such as reductases, isomerases, and synthases, then convert PGH2 to other PGs (prostaglandins) and thromboxane A2. Reactive oxygen species (ROS) produced during this biosynthetic pathway are known to contribute to tissue damage. The pro-oxidant effect of prostaglandin J2 could me mediated by its cyclopentenone ring that contains an alpha-beta-unsaturated carbonyl group that can react with sulfhydryl group of cysteine residues in glutathione and cellular proteins to inhibit ubiquitin isopeptidase activity. This may also contribute to the accumulation of ubiquitinylated proteins. This toxic positive feedback may create a self-destructive mechanism that contributes to the neurodegenerative process (FIG. 4). Neurodegenerative disorders, such as Alzheimer's disease, Parkinson's disease, and amylotropic lateral sclerosis, found to be associated with the accumulation of ubiquitinylated proteins in neuronal inclusions also exhibit signs of inflammation. Ross et al. [Trends Cell Biol., vol. 14(12):703-11 (2004)] provide a detailed discussion of the ubiquitin-proteasome pathway in Parkinson's disease and other neurodegenerative diseases. Burger et al. [Eur. J. Cancer., vol. 40(15):2217-29 (2004)] provide an insight into the ubiquitin-mediated protein degradation pathway in cancer therapeutic implications. A book edited by Peters et al, “Ubiquitin and the Biology of the Cell”, Plenum Publishing, provides information on the importance of ubiquitin in modulating cellular functions.

Thus, ubiquitin agents, such as the ubiquitin activating agents, ubiquitin conjugating agents, and ubiquitin ligating agents, are key determinants of the ubiquitin-mediated proteolytic pathway that results in the degradation of targeted proteins and regulation of cellular processes. Consequently, agents that modulate the activity of such ubiquitin agents may be used to up-regulate or down-regulate specific molecules involved in cellular signal transduction. Disease processes can be treated by such up- or down regulation of signal transducers to enhance or dampen specific cellular responses. This principle has been used in the design of a number of therapeutics, including phosphodiesterase inhibitors for airway disease and vascular insufficiency, kinase inhibitors for malignant transformation and Proteasome inhibitors for inflammatory conditions such as arthritis in the prior art.

The modulation of ubiquitin-proteasome pathway can be achieved in several manners that includes, (1) the inhibition of thioester bond formation between ubiquitin and cysteine moiety of ubiquitin activating enzyme (E1, E2, or E3), (2) the inhibition of iso-peptide bond formation between ubiquitin and lysine moiety of target protein (3) the inhibition of ubiquitin-proteasome complex, (4) acceleration of proteolysis by ubiquitin-proteasome complex (acceleration of proteasome ligase, E3, action), (5) selective inhibition of cyclooxygenase enzyme, (6) use of thiol reducing antioxidants, (7) caspase inhibitors, and (8) use of molecular chaperones to attenuate the accumulation of ubiquitinylated proteins. The molecular chaperones could thus be highly beneficial in the reduction of inflammation caused by accumulating ubiquitin-proteasome complex, which could be useful for the treatment of skin aging, inflammation, ulcer and wound healing, and enzyme malfunction related ailments, and this aspect is the focal point of the present invention.

Commercial Importance of Ubiquitin Pathway Modulators.

The discovery of the ubiquitin pathway and its many substrates and functions has revolutionized our concept of intracellular protein degradation. From an unregulated, non-specific terminal scavenger process, it has become clear that proteolysis of cellular proteins is a highly complex, temporally controlled and tightly regulated process which plays important roles in a broad array of basic cellular processes. It is carried out by a complex cascade of enzymes and displays a high degree of specificity towards its numerous substrates. Among these are cell cycle and growth regulators, components of signal transduction pathways, enzymes of house keeping and cell-specific metabolic pathways, and mutated or post-translationally damaged proteins. The system is also involved in processing major histocompatibility complex (MHC) class I antigens. For many years it has been thought that activity of the system is limited to the cytosol and probably to the nucleus. However, recent experimental evidence has demonstrated that membrane-anchored and even secretory pathway-compartmentalized proteins are also targeted by the system. These proteins must be first translocated in a retrograde manner into the cytosol, as components of the pathway have not been identified in the endoplasmic reticulum (ER) lumen. With the multiple cellular targets, it is not surprising that the system is involved in the regulation of many basic cellular processes such as cell cycle and division, differentiation and development, the response to stress and extracellular modulators, morphogenesis of neuronal networks, modulation of cell surface receptors, ion channels and the secretory pathway, DNA repair, regulation of the immune and inflammatory responses, biogenesis of organelles and apoptosis. One would also predict that aberrations in such a complex system may be implicated in the pathogenesis of many diseases, both inherited and acquired. Recent evidence shows that this is indeed the case.

Degradation of a protein by the ubiquitin system involves two distinct and successive steps: (i) covalent attachment of multiple ubiquitin molecules to the target protein; and (ii) degradation of the tagged protein by the 26S proteasome or, in certain cases, by the lysosomes/vacuole. Conjugation of ubiquitin to the substrate proceeds via a three-step mechanism. Initially, ubiquitin is activated in its C-terminal Gly by the ubiquitin-activating enzyme, E1. Following activation, one of several E2 enzymes (ubiquitin-carrier proteins or ubiquitin-conjugating enzymes, UBCs) transfers ubiquitin from E1 to a member of the ubiquitin-protein ligase family, E3, to which the substrate protein is specifically bound. This enzyme catalyzes the last step in the conjugation process, covalent attachment of ubiquitin to the substrate. The first moiety is transferred to an epsilon-NH₂ group of a Lys residue of the protein substrate to generate an isopeptide bond. The first moiety can be also conjugated in a linear mannerto the N-terminal residue of the substrate. In successive reactions, a polyubiquitin chain is synthesized by transfer of additional ubiquitin moieties to Lys48 of the previouslyconjugated molecule. The chain serves, most probably, as a recognition marker for the protease. The structure of the system appears to be hierarchical: a single E1 activates ubiquitin required for all modifications. It can transfer ubiquitin to several species of E2 enzymes, and each E2 acts with either one or several E3s. Only a few E3s have been identified so far, but it appears that these enzymes belong to a large and rapidly growing family of proteins. A major, as yet unresolved problem involves the mechanisms that underlie the high specificity and selectivity of the system. Why are certain proteins extremely stable while others are exceedingly short-lived? Why are certain proteins degraded at a particular time point in the cell cycle or only following specific extracellular stimuli, while they are stable under all other physiological conditions? It appears that specificity is determined by two distinct groups of proteins. Within the ubiquitin system, substrates are recognized by the different E3s. Some proteins are recognized via primary signals and bind directly to E3s. However, many proteins must undergo post-translational modification such as phosphorylation, or associate with ancillary proteins such as molecular chaperones prior to recognition by the appropriate ligase. Thus, the modifying enzymes and ancillary proteins also play an important role in the recognition process. As for the E3s, except for a few cases, it is not likely that each substrate is targeted by a single ligase; rather, it is conceivable that a single E3 recognizes a subset of similar, but clearly not identical, structural motifs.

Involvement of the Ubiquitin System in the Pathogenesis of Diseases.

Considering the broad range of substrates and processes in which the ubiquitin pathway is involved, it is not surprising that aberrations in the system have been implicated in the pathogenesis of several diseases, both inherited and acquired. The pathological states can be divided into two groups: (i) those that result from loss of function, a mutation in an enzyme or substrate that leads to stabilization of certain proteins; and (ii) those that result from a gain of function, resulting in accelerated degradation.

Malignancies

It has been noted that the level of p53 is extremely low in uterine cervical carcinomas caused by high-risk strains of HPV. It has been shown that the suppressor is targeted for degradation by E6-AP following formation of a ternary complex with E6-16 or 18, members of the high-risk family of HPV E6 oncoproteins. E6s derived from low-risk strains do not associate with E6-AP and do not destabilize p53. The strong correlation between sensitivity of different genetic polymorphic isotypes of p53 to E6-mediated degradation and the prevalence of cervical carcinoma in women further corroborates the direct linkage between targeting of p53 and malignant transformation. P53-Arg is significantly more susceptible to E6 targeting than p53-Pro. Accordingly, individuals homozygous for the Arg allele are 7-fold more susceptible to HPV-associated tumors than heterozygotes. Removal of the suppressor by the oncoprotein appears to be a major mechanism utilized bythe virus to transform cells. In another case it was shown that c-Jun, but not its transforming counterpart v-Jun, can be ubiquitinated and rapidly degraded. It has been shown that the gamma domain of c-Jun, a 27 amino acid sequence that is missing in the retrovirus-derived molecule, destabilizes the protein. This domain is not ubiquitinated but may serve as an anchoring site for the specific E3. The lack of the domain from v-Jun, a protein that is otherwise highly homologous to c-Jun, provides a mechanistic explanation for its stability, and possibly for its transforming activity. This is also an example of the complex mechanisms evolved by viruses to ensure continuity of replication and infection. An interesting correlation was found between low levels of p27, the G₁ CDK inhibitor whose degradation is essential for G₁S transition, and aggressive colorectal and breast carcinomas. This low level is due to specific activation of the ubiquitin system, as the p27 found in these tumors is the wild type. The strong correlation between the low level of p27 and the aggressiveness of the tumor makes p27 a powerful prognostic tool for survival. Another interesting example involves beta-catenin, which plays a major role in signal transduction and differentiation of the colorectal epithelium, and possibly in the multi-step development of the highly prevalent colorectal tumors. In the absence of signaling, glycogen synthase kinase-3 (GSK-3) is active and, via phosphorylation of a specific Ser residue, targets beta-catenin for degradation. Stimulation promotes dephosphorylation, stabilization and subsequent activation of beta-catenin via complex formation with otherwise inactive subunits of transcription regulators such as lymphocyte enhancer factor (LEF) and T-cell factor (TCF). In the cell, beta-catenin generates a complex with other proteins, including the tumor suppressor adenomatous polyposis coli (APC). The complex may be analogous to the ligase complexes cyclosome/APC and SCF (see above); here too, the identity of the ligase subunit is unknown.

Genetic Diseases

Cystic Fibrosis (CF).

The CF gene encodes the CF transmembrane regulator (CFTR), which is a chloride ion channel. Only a small fraction of the wild-type protein matures to the cell surface most of the protein is degraded from the ER by the ubiquitin system. The most frequent mutation in CFTR is F508. Despite normal ion channel function, CFTR does not reach the cell surface at all and is retained in the ER, from which it is degraded. It is possible that the rapid and efficient degradation results in complete lack of cell surface expression of the F508 protein, and contributes to the pathogenesis of the disease.

Angelman's Syndrome.

This is a rare inherited disorder characterized by mental retardation, seizures, frequent out-of-context laughter and abnormal gait. The syndrome is an example of genomic imprinting and the deleted chromosomal segment is always maternal in origin. The affected protein is the E3 enzyme E6-AP. While the target substrate of E6-AP has not been identified, elucidation of the defect clearly demonstrates an important role for the ubiquitin system in human brain development. It also shows that E6-AP has a native cellular substrate(s) targeted in the absence of E6.

Liddle Syndrome.

This is a hereditary form of hypertension that results from deletion of a proline rich (PY) region in the subunits of the heterotrimeric amiloride-sensitive ENaC. The HECT domain E3 NEDD4 binds to the PY motif of ENaC via its WW domain. ENaC is short-lived in vivo, and its side chains were shown to be ubiquitinated. Mutations affecting recognition of the channel result in its stabilization, excessive reabsorption of sodium and water, and the subsequent development of hypertension.

Immune and Inflammatory Responses

Two interesting examples involve an interaction between the ubiquitin pathway and viruses, where the viruses exploit the system to escape immune surveillance. The Epstein-Barr nuclear antigen 1 (EBNA-1) protein persists in healthy carriers for life and is the only viral protein regularly detected in all EBV-associated malignancies. Unlike EBNAs-2, 3 and 4, which are strong immunogens, EBNA-1 cannot elicit a cytotoxic T lymphocyte (CTL) response. The persistence of EBNA-1 contributes, most probably, to some of the virus-related pathologies. A long C-terminal Gly-Ala repeat was found to inhibit degradation of EBNA-1 by the ubiquitin system. Thus, the GA repeat constitutes a.cis-acting element that inhibits processing and subsequent presentation of the resulting epitopes. A second example involves the human cytomegalovirus (CMV) that encodes two ER resident proteins, US2 and US11. These proteins target MHC class I heavy-chain molecules for degradation. The MHC molecules are normally synthesized on ER-bound ribosomes and transported to the ER. In cells expressing US2 or US11, the MHC molecules are transported in a retrograde manner back to the cytoplasm, deglycosylated and degraded by the proteasome following ubiquitination. The viral products bind to the MHC molecules and escort them to the translocation machinery, where they are transported back into the cytoplasm. The virus-mediated destruction of the MHC molecules does not allow presentation of viral antigenic peptides, thus enabling the virus to evade the immune system.

Neurodegenerative Diseases

Ubiquitin immunohistochemistry has revealed enrichment in conjugates in senile plaques, lysosomes, endosomes, and a variety of inclusion bodies and degenerative fibers in many neurodegenerative diseases such as Alzheimer's (AD), Parkinson's and Lewy body diseases, amyotrophic lateral sclerosis (ALS) and Creutzfeld-Jakob disease (CJD). However, from these morphological studies it is impossible to conclude what pathogenetic role the ubiquitin system plays in these pathologies. While there can be a cell-specific defect in one of the enzymes of the system, it is more likely that an alteration in one of the protein substrates, either inherited or acquired, renders it resistant to proteolysis. Accumulation of the substrate(s) and/or of the resulting conjugates in aggregates and inclusion bodies may be toxic to the cell. Lack of animal models for most of these diseases and their long periods of development make any mechanistic approach to the problem difficult.

An interesting case involves the proteasome-mediated degradation of the cleaved, C-terminal fragment of presenilin 2. PS2 is a transmembrane protein that is probably involved in trafficking or processing of proteins between different cellular compartments. It is implicated in the transport of the amyloid precursor protein (APP) and its processing to amyloid. Mutations in PS2 and in its homologous protein, PS1, are responsible for the majority of cases of early onset AD. One mutation, N1411, is prevalent in the Volga-German type of familial AD. For normal functioning, PS2 is first cleaved and the C-terminal domain is degraded. The N-terminal domain probablyconstitutes the active form of the molecule. Proteasome inhibitors lead to accumulation of polyubiquitinated PS2, and also to accumulation of the C-terminal fragment. Introduction of the Volga-German mutation to wild-type presenilin leads to a dramatic decrease in the rate of processing of PS2, similar to that observed in proteasome inhibitor-treated cells. Thus, it appears that a defect in the processing (and possible subsequent activation) of PS2 may play a role in the pathogenesis of this form of AD. In a different example, a frame shift mutation in the ubiquitin-B gene was identified in a patient with the more prevalent nonfamilial late-onset form of AD. While it is clear that the mutation plays an important role in the pathogenesis of the disease, it is possible that a primary, so far unidentified event leads to formation of abnormal protein(s), and the lack of a functional ubiquitin system leads to their accumulation and the resulting pathology.

In Huntington disease and spinocerebellar ataxias, the affected genes, HUNTINGTIN and ATAXINS, encode proteins with various lengths of CAG/polyglutamine repeats. Recent studies have shown that these proteins aggregate in ubiquitin- and proteasome-positive intranuclear inclusion bodies. It is possible that these abnormal proteins cannot be removed by the system, and their aggregation and precipitation play a role in cell toxicity and subsequent pathologies.

Ubiquitin and Muscle Wasting

Skeletal muscle wasting, which occurs in various pathological states such as fasting, starvation, sepsis and denervation, results from accelerated ubiquitin-mediated proteolysis. The extracellular stimuli and signaling pathways that activate the ubiquitin system in response to the different pathological states are still obscure.

Diseases Associated with Animal Models

Two interesting pathological states have been described in mouse models which may also have implications for human diseases. Inactivation of HR6B, an E2 involved in DNA repair and in targeting of the N-end rule pathway and other protein substrates, leads to the single defect of male sterility due to defects in spermatogenesis. The target substrate proteins may be histones, as their degradation is critical for postmeiotic chromatin remodeling which occurs during spermatogenesis. Another interesting case is that of the Itch locus which encodes a novel E3 enzyme. Defects in the locus result in a variety of syndromes that affect the immune system. Some animals develop inflammatory disease of the large intestine. Others develop a fatal disease characterized by pulmonary interstitial inflammation, alveolar proteinosis, inflammation of the stomach and skin glands that results in severe itching and scarring, and hyperplasia of the lymphoid and hematopoietic cells. The target protein(s) of the Itch E3 is not known.

The exponential increase of information on the ubiquitin system has made it impossible to describe all the important advances in the field in a single prior art review, however comprehensive. Many recent review articles and monographs have described different aspects of the pathway. Passmore et al. [Biochem J., vol. 379(Pt 3):513-25 (2004)] provide a detailed review of some of these complex mechanisms.

Ramesh et al. (U.S. patent application ser. no. 20050009871) describe compounds, for example FIG. 5, useful as ubiquitin ligase inhibitors. The compounds of the invention are useful as inhibitors of the biochemical pathways of organisms in which ubiquitinylation is involved. The invention also provides for pharmaceutical compositions comprising the compounds described in the invention for the treatment of conditions that require inhibition of ubiquitin ligases.

FIG. 5.

Chung, K. K., Y. Zhang, et al. (2001). “Parkin ubiquitinates the alpha-synuclein-interacting protein, synphilin-1: implications for Lewy-body formation in Parkinson disease.” Nat Med 7(10):1144-50 report that Parkinson disease is a common neurodegenerative disorder characterized by the loss of dopaminergic neurons and the presence of intracytoplasmic-ubiquitinated inclusions (Lewy bodies). Mutations in alpha-synuclein (A53T, A30P) and parkin cause familial Parkinson disease. Both these proteins are found in Lewy bodies. The absence of Lewy bodies in patients with parkin mutations suggests that parkin might be required for the formation of Lewy bodies. Here we show that parkin interacts with and ubiquitinates the alpha-synuclein-interacting protein, synphilin-1. Co-expression of alpha-synuclein, synphilin-1 and parkin result in the formation of Lewy-body-like ubiquitin-positive cytosolic inclusions. We further show that familial-linked mutations in parkin disrupt the ubiquitination of synphilin-1 and the formation of the ubiquitin-positive inclusions. These results provide a molecular basis for the ubiquitination of Lewy-body-associated proteins and link parkin and alpha-synuclein in a common pathogenic mechanism through their interaction with synphilin-1.

Gasser, T. (2001). “Genetics of Parkinson's disease.” J Neurol 248(10): 833-40 report that over the past few years, several genes for monogenically inherited forms of Parkinson's disease (PD) have been mapped and/or cloned. In a small number of families with autosomal dominant inheritance and typical Lewy-body pathology, mutations have been identified in the gene for alpha-synuclein. Aggregation of this protein in Lewy-bodies may be a crucial step in the molecular pathogenesis of familial and sporadic PD. On the other hand, mutations in the parkin gene cause autosomal recessive parkinsonism of early onset. In this form of PD, nigral degeneration is not accompanied by Lewy-body formation. Parkin-mutations appear to be a common cause of PD in patients with very early onset. Parkin has been implicated in the cellular protein degradation pathways, as it has been shown that it functions as a ubiquitin ligase. The potential importance of this pathway is also highlighted by the finding of a mutation in the gene for ubiquitin C-terminal hydrolase L1 in another small family with PD. Other loci have been mapped to chromosome 2p and 4p, respectively, in a small number of families with dominantly inherited PD, but those genes have not yet been identified. These findings prove that there are several genetically distinct forms of PD that can be caused by mutations in single genes. On the other hand, there is at present no direct evidence that any of these genes have a direct role in the aetiology of the common sporadic form of PD. Epidemiological, case control, and twin studies, although supporting a genetic contribution to the development of PD, all suggest a clear familial clustering only in a minority of cases. It is therefore widely believed that a combination of interacting genetic and environmental causes may be responsible in this majority of PD-cases. However, studies of gene-environment interactions have not yet produced any convincing results. Nevertheless, the elucidation of the molecular sequence of events leading to nigral degeneration in clearly inherited cases is likely to shed light also on the molecular pathogenesis of the common sporadic form of this disorder.

Giasson, B. I. and V. M. Lee (2001). “Parkin and the molecular pathways of parkinson's disease.” Neuron 31(6): 885-8 report that Parkinson's disease (PD) is a neurodegenerative disease characterized by the selective demise of specific neuronal populations leading to impairment of motor functions. Recent genetic studies have uncovered several genes involved in inherited forms of the disease. These gene products are implicated in the biochemical pathways underlying the etiology of sporadic PD. Mutations in the parkin gene causal of autosomal recessive juvenile parkinsonism highlight that ubiquitin-mediated proteolysis may play an important role in the pathobiology of PD.

Horowitz, J. M., J. Myers, et al. (2001). “Spatial distribution, cellular integration and stage development of Parkin protein in Xenopus brain.” Brain Res Dev Brain Res 126(1): 31-41 report that Parkin is an ubiquitin-protein ligase molecule abundantly expressed in mammalian brains. Deletional mutations of Parkin protein produce a disease-related parkinsonian phenotype which is inherited with an autosomal recessive mode of transmission. To gain a greater insight into the evolutionary trajectory of the protein among vertebrate species, we describe here the (i) distribution pattern, (ii) sizing of specific fragments and (iii) embryonic development of Parkin in Xenopus laevis utilizing two antibodies to the N- and C-terminal sequence of the human Parkin protein. Parkin immunoreactivity was distributed in a heterogeneous fashion throughout the adult frog brain. The telencephalon, including the olfactory bulb, striatum and nucleus accumbens, harbored high numbers of Parkin-containing cells. High numbers of immunoreactive neurons were also present in discrete regions of the thalamus and hypothalamus. Relatively moderate expression of Parkin protein was noted in the nucleus anterodorsalis tegmenti, nucleus reticularis medius and torus semicircularis. The substantia nigra exhibited a distinctive heterogeneous pattern of Parkin-immunoreactivity, especially within presumptive dopamine neurons. The cerebellum also showed high expression of Parkin-positive material. Characterization of the subcellular distribution of the protein indicated both a cytoplasmic and nuclear integration of Parkin-immunoreactivity. This pattern of subcellular localization was similar to that observed in human brain material, perhaps reflecting distinct structural phosphorylation sites of the Parkin protein. Western blot analysis identified three specific bands with molecular weights varying from 50 to 65 kDa in adult Xenopus brain. However, studies on the temporal expression of Parkin during development showed a complete absence of cellular immunoreactivity which was especially conspicuous during late premetamorphic stages of frog development. These results suggest that the ubiquitination activity of Parkin is limited or non-existent during embryogenesis, but appears to assume a more functional role during adulthood as reflected by the high distribution pattern of the protein within major circuits of the amphibian brain.

Klein, C. (2001). “[The genetics of Parkinson syndrome].” Schweiz Rundsch Med Prax 90(23): 1015-23 report that a genetic contribution to the etiology of Parkinson's disease was first suspected by Charcot and later confirmed by case control, family, and twin studies, as well as by the description of large parkinsonian families with Mendelian inheritance of the disease. Recent progress in the field of molecular neurogenetics has led to the identification of several Parkinson disease genes and gene loci. Mutations in the alpha-Synuclein gene (PARK1) and in the gene for the ubiquitin C-terminal hydrolase I (PARK5), along with two gene loci harboring currently unknown genes (PARK3 and PARK4), have been linked to very rare autosomal dominantly inherited parkinsonian syndromes. Mutations in the parkins gene (PARK2), causing autosomal recessive early-onset parkinsonism, are much more common and therefore of clinical relevance. A second gene locus for an autosomal dominantly inherited Parkinsonian syndrome was recently localized on chromosome 1 (PARK6). All three parkinson genes identified thus far imply the involvement of the ubiquitin pathway of protein degradation in the pathogenesis of Parkinson's disease.

Layfield, R., A. Alban, et al. (2001). “The ubiquitin protein catabolic disorders.” Neuropathol Appl Neurobiol 27(3): 171-9 report that the ubiquitin-proteasome system of intracellular proteolysis is essential for cell viability. We propose the concept that neurodegenerative diseases such as Alzheimer's and Parkinson's, as well as other conditions including some types of cancer, collectively represent a raft of ubiquitin protein catabolic disorders' in which altered function of the ubiquitin-proteasome system can cause or directly contribute to disease pathogenesis. Genetic abnormalities within the ubiquitin pathway, either in ubiquitin-ligase (E3) enzymes or in deubiquitinating enzymes, cause disease because of problems associated with substrate recognition or supply of free ubiquitin, respectively. In some cases, mutations in protein substrates of the ubiquitin-proteasome system may directly contribute to disease progression because of inefficient substrate recognition. Mutations in transcripts for the ubiquitin protein itself (as a result of ‘molecular misreading’) also affect ubiquitin-dependent proteolysis with catastrophic consequences. This has been shown in Alzheimer's disease and could apply to other age-associated neurodegenerative conditions. Within the nervous system, accumulation of unwanted proteins as a result of defective ubiquitin-dependent proteolysis may contribute to aggregation events, which underlie the pathogenesis of several major human neurodegenerative diseases.

Lev, N. and E. Melamed (2001). “Heredity in Parkinson's disease: new findings.” Isr Med Assoc J 3(6): 435-8 report that multiple factors have been hypothesized over the last century to be causative or contributory for Parkinson's disease. Hereditary factors have recently emerged as a major focus of Parkinson's disease research. Until recently most of the research on the etiology of Parkinson's disease concentrated on environmental factors, and the possibility that genetic factors contribute significantly to the pathogenesis of Parkinson's disease has been neglected. However, it has become increasingly apparent that even in sporadic cases, the disease most likely reflects a combination of genetic susceptibility and an unknown environmental insult. Moreover, the identification of genes and proteins that may cause hereditary parkinsonism substantially contributes to our ability to understand the pathogenesis of Parkinson's disease and may help in the early identification of the disease and its treatment. The discovery of alpha-synuclein mutations in families with autosomal dominant Parkinson's disease sheds light on its role in sporadic Parkinson's disease. It seems that this protein tends to aggregate when the cellular milieu is altered [14-16]. The question as to the exact changes that cause its deposition remains open. One of the major possibilities is oxidative stress [16]. The role of these aggregates in neuronal cell death is also still unclear. Transgenic mice expressing wild-type human alpha-synuclein developed progressive accumulation of alpha-synuclein and ubiquitin-immunoreactive inclusions in neurons in the neocortex, hippocampus and the substantia nigra. These alterations were associated with loss of dopaminergic terminals and motor impairments [24]. This finding suggests that accumulation of alpha-synuclein may play a causal role in sporadic Parkinson's disease as well. The parkin protein seems to be a crucial survival factor for nigral neurons [15]. The parkin protein is related to the ubiquitin pathway, which is important in the elimination of damaged proteins. Ubiquitin-mediated degradation of proteins plays a central role in the control of numerous processes, including signal transduction, receptor and transcriptional regulations, programmed cell death, and breakdown of abnormal proteins that may interfere with normal cell functions. Further studies on the function of Parkin protein and its relation to the ubiquitin pathway could elucidate at least one of the molecular mechanisms of nigral neuronal death. A mutation in the ubiquitin carboxy-teminal hydrolase L1 gene also implies the importance of the ubiquitin pathway in Parkinson's disease. Abnormal tau protein was found to be the cause of familial frontotemporal dementia and parkinsonism. It tends to form filamentous structures, which may lead to neuronal death. Elucidation of the molecular mechanism of neuronal death in this disease may contribute to our understanding of sporadic diseases with tau accumulation, such as corticobasal degeneration, progressive supranuclear palsy, Pick's disease, Alzheimer's disease and possibly also the pathogenesis of Parkinson's disease. Other genetic loci have been identified by linkage analysis of patients with familial parkinsonism. These loci conceal other genes and proteins that may be pivotal factors in the pathogenesis of Parkinson's disease. The discovery of genetic mutations in patients with parkinsonism may offer us new insights into the understanding of the pathways leading to neuronal death and development of Parkinson's disease. It may also help in the early identification of susceptible people to this disease and possibly in developing new treatment strategies.

Levecque, C., A. Destee, et al. (2001). “No genetic association of the ubiquitin carboxy-terminal hydrolase-L1 gene S18Y polymorphism with familial Parkinson's disease.” J Neural Transm 108(8-9): 979-84 report that Parkinson's disease (PD) is a neurodegenerative disorder for which genetic susceptibility has been documented in sporadic and familial cases. Recently, a polymorphism located in exon 3 at codon 18 (S18Y) of the Ubiquitin Carboxy-terminal Hydrolase-L1 (UCH-L1) gene has been associated with the disease in 2 populations of German origin and also in ajapanese population. We tested the impact of this polymorphism in a French sample of familial PD patients (n=114) and controls (n=93). No association was observed, indicating that this polymorphism did not confer susceptibility for familial PD in our population, even among the youngest age of onset group. This observation suggests that the previous positive results obtained may reflect mechanisms restricted to the sporadic form of the disease or to a founder effect of the disease susceptibility.

McNaught, K. S. and P. Jenner (2001). “Proteasomal function is impaired in substantia nigra in Parkinson's disease.” Neurosci Lett 297(3): 191-4 report that the accumulation of alpha-synuclein, ubiquitin and other proteins in Lewy bodies in degenerating dopaminergic neurones in substantia nigra in idiopathic Parkinson's disease (PD) suggest that inhibition of normal/abnormal protein degradation may contribute to neuronal death. We now show for the first time that the chymotrypsin-(39%), trypsin-(42%) and postacidic-like (33%) hydrolysing activities of 20/26S proteasome are impaired in substantia nigra in PD. Proteasome inhibition does not appear to result from drug treatment since high concentrations of L-3,4-dihydroxyphenylalanine had no effect on enzymatic activity in vitro. These observations provide the first direct evidence that inhibition of the ubiquitin-proteasome pathway leading to altered protein handling and Lewy body formation may be responsible for degeneration of the nigrostriatal pathway in idiopathic PD. McNaught, K. S., C. W. Olanow, et al. (2001). “Failure of the ubiquitin-proteasome system in Parkinson's disease.” Nat Rev Neurosci 2(8): 589-94, and Mizuno, Y., N. Hattori, et al. (2001). “Parkin and Parkinson's disease.” Curr Opin Neurol 14(4): 477-82 report that Parkin is the causative gene for an autosomal recessive form of Parkinson's disease. The gene was discovered in 1998. The parkin gene is a novel gene containing 12 exons spanning over 1.5 Mb and encodes a protein of 465 amino acids with a molecular mass of approximately 52,000 M(r). Various deletion mutations and point mutations have been discovered in patients with autosomal recessive Parkinson's disease. The substantia nigra and the locus coeruleus selectively undergo neurodegeneration without forming Lewy bodies. The parkin gene product, Parkin protein, has a unique structure with a ubiquitin-like domain in the amino-terminus and a RING finger motif in the carboxy terminus. The function of Parkin was not known until recently. During the year 2000, great progress was made in defining its function. First of all, Parkin was found to be a ubiquitin-protein ligase (E3), a component of the ubiquitin system, which is an important adenosine triphosphate-dependent protein degradation machinery. In addition, CDCrel-1, a synaptic vesicle associated protein, was found to be a substrate for Parkin as an E3. Although many studies still need to be performed to elucidate the molecular mechanism of the selective nigral neurodegeneration in this form of familial Parkinson's disease, it will not be too long before this is accomplished. In this review article, we evaluate the developments in this area published since 1 Feb. 2000.

Shastry, B. S. (2001). “Parkinson disease: etiology, pathogenesis and future of gene therapy.” Neurosci Res 41(1): 5-12 report that Parkinson disease (PD) is a progressive neurological disorder with a prevalence of 1-2% in people over the age of 50. It has a world-wide distribution and has no gender preference. The neurological hallmark of PD is the presence of Lewy bodies and is characterized by the degeneration of nigrostriatal dopaminergic neurons. The causes of PD are unknown but considerable evidence suggests a multifactorial etiology involving genetic and environmental factors. A molecular genetic approach identified three genes and at least two additional loci in rare familial forms of PD. Two of these genes are involved in the ubiquitin mediated pathway of protein degradation and the third one is a highly expressed protein in the synaptic terminal and is called alpha-synuclein. In animal models, it has been shown that use of the household pesticide which is known to contain rotenone, causes PD. Thus, a combined action of genetic and environmental factors is responsible for the pathogenesis of PD. Although use of levodopa or dopamine agonists can substantially reduce clinical symptoms, and transplantation of fetal nerve tissue still remains as an alternative therapy (although it has been recently shown to be having no overall benefit), directed delivery of glial cell derived neurotrophic factor (known to have trophic effects on dopaminergic neurons) may also be a beneficial therapeutic option for PD patients.

Shimura, H., M. G. Schlossmacher, et al. (2001). “Ubiquitination of a new form of alpha-synuclein by parkin from human brain: implications for Parkinson's disease.” Science 293(5528): 263-9 report that Parkinson's disease (PD) is a common neurodegenerative disorder characterized by the progressive accumulation in selected neurons of protein inclusions containing alpha-synuclein and ubiquitin. Rare inherited forms of PD are caused by autosomal dominant mutations in alpha-synuclein or by autosomal recessive mutations in parkin, an E3 ubiquitin ligase. We hypothesized that these two gene products interact functionally, namely, that parkin ubiquitinates alpha-synuclein normally and that this process is altered in autosomal recessive PD. We have now identified a protein complex in normal human brain that includes parkin as the E3 ubiquitin ligase, UbcH7 as its associated E2 ubiquitin conjugating enzyme, and a new 22-kilodalton glycosylated form of alpha-synuclein (alphaSp22) as its substrate. In contrast to normal parkin, mutant parkin associated with autosomal recessive PD failed to bind alphaSp22. In an in vitro ubiquitination assay, alphaSp22 was modified by normal but not mutant parkin into polyubiquitinated, high molecular weight species. Accordingly, alphaSp22 accumulated in a non-ubiquitinated form in parkin-deficient PD brains. We conclude that alphaSp22 is a substrate for parkin's ubiquitin ligase activity in normal human brain and that loss of parkin function causes pathological alphaSp22 accumulation. These findings demonstrate a critical biochemical reaction between the two PD-linked gene products and suggest that this reaction underlies the accumulation of ubiquitinated alpha-synuclein in conventional PD. Stefanis, L., K. E. Larsen, et al. (2001). “Expression of A53T Mutant But Not Wild-Type alpha-Synuclein in PC12 Cells Induces Alterations of the Ubiquitin-Dependent Degradation System, Loss of Dopamine Release, and Autophagic Cell Death.” J Neurosci 21(24): 9549-9560 report that alpha-Synuclein mutations have been identified in certain families with Parkinson's disease (PD), and alpha-synuclein is a major component of Lewy bodies. Other genetic data indicate that the ubiquitin-dependent proteolytic system is involved in PD pathogenesis. We have generated stable PC12 cell lines expressing wild-type or A53T mutant human alpha-synuclein. Lines expressing mutant but not wild-type alpha-synuclein show: (1) disruption of the ubiquitin-dependent proteolytic system, manifested by small cytoplasmic ubiquitinated aggregates and by an increase in polyubiquitinated proteins; (2) enhanced baseline nonapoptotic death; (3) marked accumulation of autophagic-vesicular structures; (4) impairment of lysosomal hydrolysis and proteasomal function; and (5) loss of catecholamine-secreting dense core granules and an absence of depolarization-induced dopamine release. Such findings raise the possibility that the primary abnormality in these cells may involve one or more deficits in the lysosomal and/or proteasomal degradation pathways, which in turn lead to loss of dopaminergic capacity and, ultimately, to death. These cells may serve as a model to study the effects of aberrant alpha-synuclein on dopaminergic cell function and survival.

Tanaka, Y., S. Engelender, et al. (2001). “Inducible expression of mutant alpha-synuclein decreases proteasome activity and increases sensitivity to mitochondria-dependent apoptosis.” Hum Mol Genet 10(9): 919-26 report that Parkinson's disease (PD) is a common progressive neurodegenerative disorder caused by the loss of dopaminergic neurons in the substantia nigra. Although mutations in alpha-synuclein have been identified in autosomal dominant PD, the mechanism by which dopaminergic neural cell death occurs remains unknown. Proteins encoded by two other genes in which mutations cause familial PD, parkin and UCH-L1, are involved in regulation of the ubiquitin-proteasome pathway, suggesting that dysregulation of the ubiquitin-proteasome pathway is involved in the mechanism by which these mutations cause PD. We established inducible PC12 cell lines in which wild-type or mutant alpha-synuclein can be de-repressed by removing doxycycline. Differentiated PC12 cell lines expressing mutant alpha-synuclein showed decreased activity of proteasomes without direct toxicity. Cells expressing mutant alpha-synuclein showed increased sensitivity to apoptotic cell death when treated with sub-toxic concentrations of an exogenous proteasome inhibitor. Apoptosis was accompanied by mitochondrial depolarization and elevation of caspase-3 and -9, and was blocked by cyclosporin A. These data suggest that expression of mutant alpha-synuclein results in sensitivity to impairment of proteasome activity, leading to mitochondrial abnormalities and neuronal cell death.

Majetschak et al. (U.S. patent application ser no. 20040037822) disclose Compositions and methods for suppressing the immune system of a mammal using ubiquitin and derivatives and analogs thereof.

Bosse (U.S. patent application ser no. 20030114724) discloses certain polyubiquitin based hydrogels.

Bosse (U.S. Pat. No. 6,881,789) discloses certain polyubiquitin based hydrogels.

Fujiwara et al. (U.S. Pat. No. 6,562,947) disclose certain human skeletal muscle-specific ubiquitin-conjugating enzymes. The use of the genes makes it possible to detect the expression of the same in various tissues, analyze their structures and functions, and produce the human proteins encoded by the genes by the technology of genetic engineering. Through these, it becomes possible to analyze the corresponding expression products, elucidate the pathology of diseases associated with the genes, for example hereditary diseases and cancer, and diagnose and treat such diseases.

Garbarino et al. (U.S. Pat. No. 6,448,391) disclose certain stabilized ubiquitin-lytic peptide fusion polypeptides and a method of making the same by sub-cloning nucleic acid sequences coding for lytic peptides into a plasmid vector comprising a promoter and ubiquitin polypeptide coding sequence, wherein the ubiquitin polypeptide sequence is linked to the 5′ end of the lytic peptide nucleic acid sequence and is translated as a fusion polypeptide. These are useful as gene promoters.

Coffino et al. (U.S. Pat. No. 6,217,864) disclose a method for in vivo or ex vivo targeted degradation of intracellular proteins in situ. In particular, the invention concerns the method for degradation of intracellular proteins by inducing in cells the production of a dual-function protein containing a domain that directs a selective degradation of targeted proteins to which it is attached as well as a domain that acts as a linker between the dual-function protein and the target protein. The protein degradation directing domain is a subregion within 97 amino acids that corresponds to the N-terminus of protein antizyme (NAZ). The invention further concerns a method for inducing in vivo or ex vivo in cells the production of the dual-function protein consisting of NAZ and linker directed to destruction of specific cellular target proteins.

Vierstra et al. (U.S. Pat. No. 5,851,791) disclose a novel class of fusion proteins based on the ubiquitin-conjugating enzyme, or E2, is described. The fusion proteins include, in addition to the E2 activity, a protein binding ligand having a specific affinity for a target protein. It has been discovered that under cytosolic conditions, such E2 fusions will add a ubiquitin moiety to a target protein. Since ubiquitin addition triggers the endogenous cellular protein degradation pathway, such E2 fusion proteins can be used to selectively target proteins in a host for degradation. Thus, E2 fusion proteins genes can be introduced into transgenic organisms to defeat or inhibit natural activities or traits. The E2 fusion proteins can also be used by introduction into hosts for similar effects.

Bachmair et al. (U.S. Pat. No. 5,196,321) disclose methods of designing or modifying protein structure at the protein or genetic level to produce specified amino-termini in vivo or in vitro are described. The methods can be used to alter the metabolic stability and other properties of the protein or, alternatively, to artificially generate authentic amino-termini in proteins produced through artificial means. The methods are based upon the introduction of the use of artificial ubiquitin-protein fusions, and the discovery that the in vivo half-life of a protein is a function of the amino-terminal amino acid of the protein.

Shang et al. [J Biol Chem. 2005, Mar 24] report K6-biotinylated ubiquitin inhibited ubiquitin-dependent proteolysis, as conjugates formed with K6-biotinylated ubiquitin were resistant to proteasomal degradation. These results show that K6-modified ubiquitin is a potent and specific inhibitor of ubiquitin-mediated protein degradation.

Yoshimoto et al. [FEBS Lett., vol 579(5), 197-202 (2005)] report L-dopa and dopamine enhance the formation of aggregates under proteasome inhibition in PC12 cells.

A recent search on National Library of Medicine's (Medline) for “Ubiquitins” revealed over 7000 publications. These prior art references can not all be included in the present invention. However, it does signal the commercial importance of this technology.

As can be noted from the above prior art examples, a satisfactory and practical solution to this problem has not been achieved by any of such prior art methods. The present invention achieves the modulation of ubiquitin-proteasome pathway via novel molecular chaperones. The molecular chaperones of the present invention perform attenuation of the accumulation of ubiquitinylated proteins. Such ubiquitinylation attenuating molecular chaperones have not been disclosed in the prior art.

### DETAILED DISCUSSION

In one aspect, the present invention provides a compound that is a ubiquitinylation inhibitor. Although the exact mechanism of this process is not clearly understood at this time, it is hypothesized that ubiquitinylation inhibition is achieved by a novel binding of the molecular chaperones of the present invention with the amino group of lysine moiety of target protein, thus inhibiting, (1) the formation of ubiquitinylated target protein, and (2) the formation of ubiquitin-proteasome complex.

In another aspect, this invention provides compounds that prevent inflammation, cyclooxygenase up-regulation, and prostaglandin PGJ accumulation, and other similar disorders caused by the accumulation of ubiquitin-proteasome complexes.

In yet another aspect, this invention provides certain ubiquitinylation modulating molecular chaperone compounds and their use in the prevention and/or treatment of ailments associated with the dysfunction of ubiquitin-proteasome pathway, including wound healing, skin aging, body enzyme dysfunction, neurodegenerative disorders, and cellular apoptosis.

In yet another aspect, this invention provides molecular chaperones to attenuate the accumulation of ubiquitinylated proteins in the intracellular matrix of mammals in a cosmetically or pharmaceutically acceptable delivery system or carrier base composition.

In yet another aspect, this invention provides molecular chaperones to attenuate the accumulation of ubiquitinylated proteins for the reduction of inflammation in a cosmetically or pharmaceutically acceptable delivery system or carrier base composition.

In yet another aspect, this invention provides molecular chaperones to attenuate the accumulation of ubiquitinylated proteins for the treatment of skin aging, ulcer, wound healing, and enzyme malfunction related ailments in mammals in a cosmetically or pharmaceutically acceptable delivery system or carrier base composition.

The molecular chaperones of the present invention are specific to certain chemical structural types, a detailed description of which follows.

(1) The molecular chaperone compounds comprising; (i) At least one hydroxyaryl, polyhydroxyaryl, or N-heterocyclic compound that contains an alkyl carbon side chain with a —C═C— group directly attached to the aromatic ring.

(2) The molecular chaperone compound comprising; (i) At least one hydroxyaryl, polyhydroxyaryl, or N-heterocyclic compound that contains an alkyl carbon side chain with a —C=0 (ketone) group directly attached to the aromatic ring. The chemical structure backbone of such compounds is further illustrated in FIG. 6.

FIG. 6.

(3) The molecular chaperone compound comprising; (i) At least one hydroxyaryl, polyhydroxyaryl, or N-heterocyclic compound that contains an alkyl carbon side chain with a —CH(R)—C═C— group attached to the aromatic ring via —CH(R)— moiety. The chemical structure backbone of such compounds is further illustrated in FIG. 7, and these are selected from Honokiol, Magnolol, Shikonin, substituted Shikonins, Licoricidin, Glycyrol, Iso-Glycyrol, Anhydro-Alkannin, Mangostin, and combinations thereof.

FIG. 7.

(4) The molecular chaperone compound comprising; (i) At least one hydroxyaryl, polyhydroxyaryl, or N-heterocyclic compound that contains an alkyl carbon side chain with a —C(═O)—C═C— group attached to an aromatic ring.

(5) The molecular chaperone compound comprising; (i) At least one hydroxyaryl, polyhydroxyaryl, or N-heterocyclic compound that contains an alkyl carbon side chain with a —C═C— group as part of another aromatic ring.

The examples of the above mentioned chemical structural backbones are further described below.

(1) The molecular chaperone compound comprising; (i) At least one hydroxyaryl, polyhydroxyaryl, or N-heterocyclic compound that contains an alkyl carbon side chain with a —C═C— group directly attached to aromatic ring is selected from Resveratrol, Polydatin, Rhapontin, Hypericin, and combinations thereof. The chemical structure of some of these is shown in FIG. 8, which also serves to show their relationship with the chemical backbone criteria mentioned herein.

FIG. 8.

(2) The molecular chaperone compound comprising; (i) At least one hydroxyaryl, polyhydroxyaryl, or N-heterocyclic compound that contains an alkyl carbon side chain with a —C═O (ketone) group directly attached to aromatic ring is selected from hydroxy or polyhydroxy acetophenones, or hydroxy or Polyhydroxy propiophenones, and their variously substituted derivatives.

(3) The molecular chaperone compound comprising; (i) At least one hydroxyaryl, polyhydroxyaryl, or N-heterocyclic compound that contains an alkyl carbon side chain with a —C═O (ketone) group directly attached to aromatic ring is selected from Ellagic acid, Hypericin, and combinations thereof. The chemical structure is shown in FIG. 9, which also serves to show the relationship with the chemical backbone criteria mentioned herein.

FIG. 9.

(4) The molecular chaperone compound comprising; (i) At least one hydroxyaryl, polyhydroxyaryl, or N-heterocyclic compound that contains an alkyl carbon side chain with a —C(═O)—C═C— group attached to aromatic ring is selected from Rosmarinic acid, Curcumin, Curcuminoids, Shikonin, Ferulic acid, Ginkgetin, Morin, Mulberrin, Kuraridin, Baicalin, Isoliquiritin, Rutin, Chlorogenic acid, and combinations thereof. The chemical structure of some of these is shown in FIG. 10, which also serves to show their relationship with the chemical backbone criteria mentioned herein.

FIG. 10.

(5) The molecular chaperone compound comprising; (i) At least one hydroxyaryl, polyhydroxyaryl, or N-heterocyclic compound that contains an alkyl carbon side chain with a —C═O (ketone) group directly attached to aromatic ring is selected from hydroxy or polyhydroxy acetophenones, or hydroxy or Polyhydroxy propiophenones, hydroxy or polyhydroxy acetophenone compound is further selected from Phloridzin, Resacetophenone, Quinacetophenone, Paeonol, 2-hydroxyacetophenone, 3-hydroxyacetophenone, 4-hydroxyacetophenone, 2,3-dihydroxyacetophenone, 2,4-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, 2-Acetyl resorcinol, 4-Acetyl resorcinol, 3,4-Dihydroxyacetophenone, acetyl quinol, 1-(3-Hydroxy-4-methoxy-5-methylphenyl) ethanone, 1-(3-hydroxy-4-methoxyphenyl) ethanone, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, and combinations thereof. The chemical structure of some of these is shown in FIG. 11, which also serves to show their relationship with the chemical backbone criteria mentioned herein.

FIG. 11.

(6) The molecular chaperones to attenuate the accumulation of ubiquitinylated proteins in the intracellular matrix of mammals in a cosmetically or pharmaceutically acceptable delivery system or carrier base composition, in which cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

### EXAMPLES

The following examples are presented to illustrate presently preferred practice thereof. These examples also include the formulation of consumer desirable lotion, cream, and other such compositions for their retail marketing. As illustrations they are not intended to limit the scope of the invention. All quantities are in weight %.

### Example 1

### Ubiquitin-Ptoteasome Inhibiting Serum

Ingredients (1) Deionized water 20.0 (2) Phloridzin 5.0 (3) Methylpropanediol 69.5 (4) Dimethicone copolyol 4.0 (5) Preservatives 0.5 (6) Ammonium Acryloyidimethyltaurate/vp copolymer 1.0. Procedure. Make main batch by mixing (2) to (5) at room temperature. Pre-mix (1) and (6) to a clear paste and add to main batch with mixing. The product has a clear to slightly hazy syrup-like appearance, typical of a skin serum product. It is absorbed rapidly with a silky smooth skin feel.

### Example 2

### Ubiquitin Modulating Wound Healing Serum with Ubiquitin Modulators

Ingredients (1) Deionized water 20.0 (2) 2,6-Dihydroxy acetophenone 5.0 (3) Methylpropanediol 69.0 (4) Dimethicone copolyol 4.0 (5) Preservatives 0.5 (6) Copper Gluconate 0.5. (7) Ammonium Acryloyidimethyltaurate/VP copolymer 1.0 Procedure. Make main batch by mixing (2) to (6) at room temperature. Pre-mix (1) and (7) to a clear paste and add to main batch with mixing. The product has a clear to slightly hazy syrup-like light blue appearance, typical of a skin serum product. It is absorbed rapidly with a silky smooth skin feel.

### Example 3

### Ubiquitin Modulating Wound Healing Cream

Ingredients (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Resveratrol 1.0 (7) Ellagic acid 2.0 (8) Paeonol 1.0 (9) Preservatives 0.5. Procedure. Mix 1 to 5 and heat to 75-80° C. Adjust pH to 4.0 4.5. Cool to 35-40 C with mixing. Add 6 to 9 with mixing. Adjust pH to 4.0-4.5, if necessary. White to off-white cream.

### Example 4

### Ubiquitin Modulating Collagen Boosting Antiaging Facial Mask Composition

Ingredients. (1) Chitosan 5.0 (2) Honokiol 5.0 (3) Glycerin 17.7 (4) Water 70.6 (5) Rosmarinic acid 0.5 (6) Niacinamide Lipoate 0.5 (7) Glutathione 0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck and left for 10 to 30 minutes, then rinsed off.

### Example 5

### Ubiquitin Modulating Skin Discoloration and Age Spots Cure Cream

Ingredients (1) Water 65.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (6) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Niacinamide Lactate 1.0 (16) Phloridzin 3.1 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Polysorbate-20 2.0 (23) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained.

### Example 6

### Ubiquitin Modulating Anti-Inflammatory Cream

Ingredients (1) Water 62.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (6) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Osthol 4.0 (16) Hypericin 2.2 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Pyridoxine Salicylate (23) Polysorbate-20 2.0 (24) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained.

### Example 7

### Ubiquitin Modulating Anti-Inflammatory Skin Cleanser

Ingredients (1) PEG-6 63.329 (2) Hydroxypropyl Cellulose 0.3 (3) Boswellia Serrata 0.05 (4) Sodium Cocoyl Isethionate 20.0 (5) Sodium Lauryl Sulfoacetate 5.0 (6) L-Glutathione 0.01 (7) Resveratrol 0.01 (8) 2,5-Dihydroxy Acetophenone 0.1 (9) 2,6-Dihydroxy Acetophenone 0.001 (10) Ascorbic acid 10.0 (11) Phenoxyethanol 0.7 (12) Ethylhexylglycerin 0.3 (13)Fragrance 0.2. Procedure. Mix (1) and (2) to a clear thin gel. Add all other ingredients and mix in a homogenizer. A white cream-like cleanser is obtained.

### Example 8

### Ubiquitin Modulating Arthritis Anti-Inflammatory Gel

Ingredients. (1) C12-15 Alkyl Benzoate 67.75 (2) Ethylenediamine/Hydrogenated Dimer Dilinoleate Copolymer Bis-Di-C14-18 Alkyl Amide 10.0 (3) Ximenia Oil 0.1 (4) Capsaicin 0.25 (5) Magnolol (and) Honokiol) 0.2 (6) Paeonol 0.5 (7) Ellagic acid 0.2 (8) Zeolite 20.0 (9) Fragrance 1.0. Procedure. Mix (1) and (2) and heat at 80 to 90 C till clear. Cool to 40 to 50 C and add all other ingredients and mix. Cool to room temperature. A white gel-like product is obtained.

### Example 9

### Ubiquitin Modulating Anti-Inflammatory Transparent Gel

Ingredients. (1) C12-15 Alkyl Benzoate 96.75 (2) Dibutyl Lauroyl Glutamide 1.0 (3) Ximenia Oil 0.1 (4) Capsaicin 0.25 (5) Magnolol (and) Honokiol 0.2 (6) Paeonol 0.5 (7) Tetrahydrocurcuminoids 0.2 (8) Fragrance 1.0. Procedure. Mix (1) and (2) and heat at 95 to 110 C till clear. Cool to 40 to 50 C and add all other ingredients and mix. Cool to room temperature. A transparent gel-like product is obtained.

### Example 10

### Topical Anesthetic Spray Lotion with Anti-Inflammatory Ubiquitin Modulating Agents

Ingredients (1) PEG-481.0 (2) Benzocaine 16.0 (3) Fragrance 0.5 (4) Paeonol 0.5 (5) Osthol 2.0. Procedure. Mix all ingredients till a clear solution is obtained. Fill in spray bottles.

### Example 11

### Ubiquitin Modulating Anti-Inflammatory Color-Changing Mask with Controlled Release

Ingredients (1) Grapeseed oil 34.28 (2) Ethylenediamine/Hydrogenated Dimer Dilinoleate Copolymer Bis-Di-C14-18 Alkyl Amide 5.0 (3) Dimthicone 2.0 (4) Propyl Paraben 0.3 (5)Jojoba oil 0.5 (6) Sweet Almond oil 4.0 (7) Shea butter 0.2 (8) Mango butter 0.2 (9) Avocado utter 0.2 (10) Murumuru butter 0.2 (11) Color Change Green/Blue dye 0.01 (12) Baicalin 5.5 (13) Vitamin E 0.11 (14) Phenoxyethanol 0.7 (15) Zeolite 31.0 (16) Ethylhexylglycerin 0.5 (17) Laureth-3 15.0 (18) Fragrance 0.5. Procedure. Mix (1) to (10) and heat at 70 to 80 C till clear. Cool to 35 to 45 C and all other ingredients and mix. Cool to room temperature. A light green thin paste is obtained. Upon contact with water, it turns blue and releases heat.

### Example 12

### Ubiquitin Modulating Shampoo

Ingredients. (1) Water 64.2 (2) Mulberrin (1.2) (3) Sodium Lauryl Sulfoacetatel 0.0 (4) Disodium Laureth Sulfosuccinate 20.0 (5) Phenoxyethanol 0.7 (6) Chlorphenesin 0.3 (7) PEG-120 Methyl Glucose Dioleate 2.5 (8) Hydrolyzed Soy Protein 0.5 (9) Hydrolyzed Silk Protein 0.5 (10) Oat Extract 0.1. Procedure. Mix (1) to (7) and heat at 60 to 70 C to a clear solution. Cool to 35 to 40 C and add all other ingredients and mix. Cool to room temperature.

### Example 13

### Ubiquitin Modulating Topical Inflammation Control Massage Lotion

Ingredients (1) Water 39.158 (2) Acrylates/C10-30 Alkyl Acrylate Crosspolymer 0.5 (3) Escin 0.1 (4) Sodium Stearyl Phthalamate 1.0 (5) Sodium Hydroxide 0.142 (6) Cetyl Alcohol 4.0 (7) Phenoxyethanol 0.7 (8) Chlorphenesin 0.3 (9) Grapeseed oil 10.0 (10) Ethylhexylglycein 0.5 (11) Polysorbate-20 10.0 (12) PEG-6 2.0 (13) Baicalin 0.1 (14) Magnolol 0.1 (15) Paeonol 0.2 (16) Fragrance 1.0. Procedure. Mix (1) to (11) and heat at 80 to 90 C till clear. Cool to 45 to 55. Pre-mix (12) to (16) and add to main batch and mix. Cool to room temperature and adjust pH to 7.5.

### Example 14

### Ubiquitin Modulating Anti-Inflammatory Make-Up Remover Fluid

Ingredients (1) Water 39.158 (2) Acrylates/C10-30 Alkyl Acrylate Crosspolymer 0.5 (3) Harpagoside 0.1 (4) Sodium Stearyl Phthalamate 1.0 (5) Sodium Hydroxide 0.142 (6) Cetyl Alcohol 4.0 (7) Phenoxyethanol 0.7 (8) 1,2-Octanediol 0.3 (9) Grapeseed oil 10.0 (10) Methyl Soyate 30.0 (11) Ethylhexylglycein 0.5 (12) Polysorbate-20 10.0 (13) PEG-6 2.0 (14) Tetrahydrocurcuminoids 0.1 (15) Magnolol 0.1 (16) Paeonol 0.2 (17) Fragrance 1.0. Procedure. Mix (1) to (12) and heat at 80 to 90 C till clear. Cool to 45 to 55. Pre-mix (13) to (16) and add to main batch and mix. Add (17) and mix. Cool to room temperature and adjust pH to 7.5.

### DESCRIPTION OF THE DRAWINGS

FIG. 1. Amino Acid Sequence of Ubiquitin.

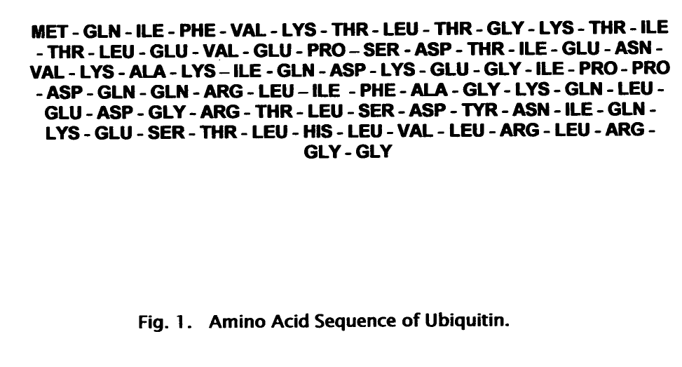

FIG. 2. Iso-peptide Chemical Bond.

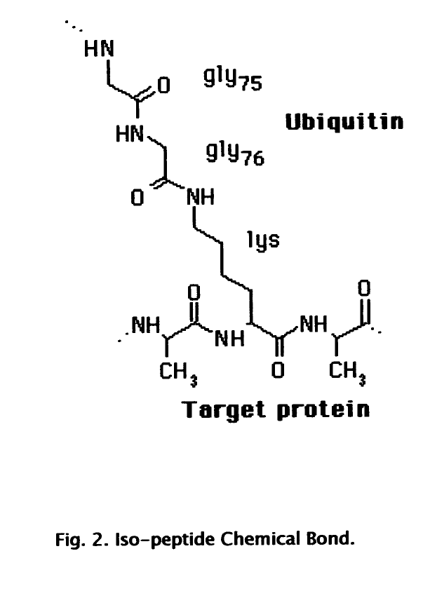

FIG. 3. Ubiquitin-Proteasome Complex.

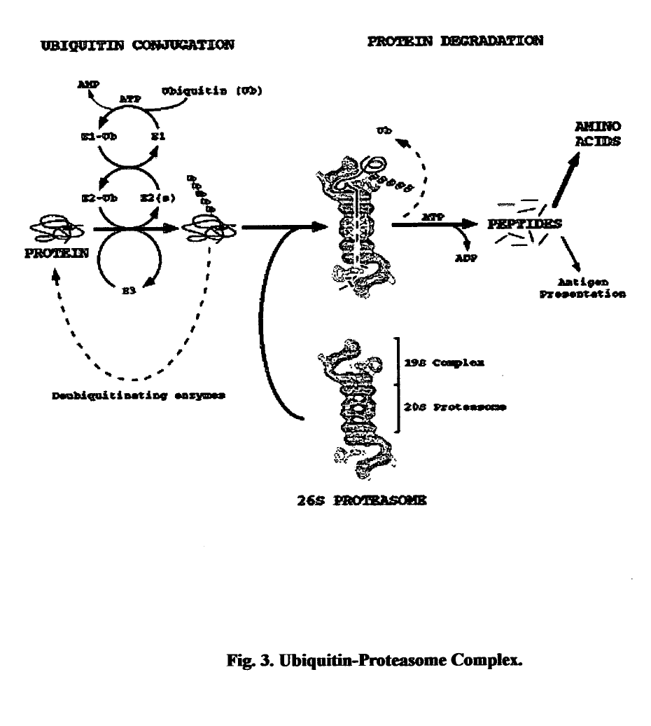

FIG. 4. Ubiquitin- Cyclooxygenase Up-rergulation.

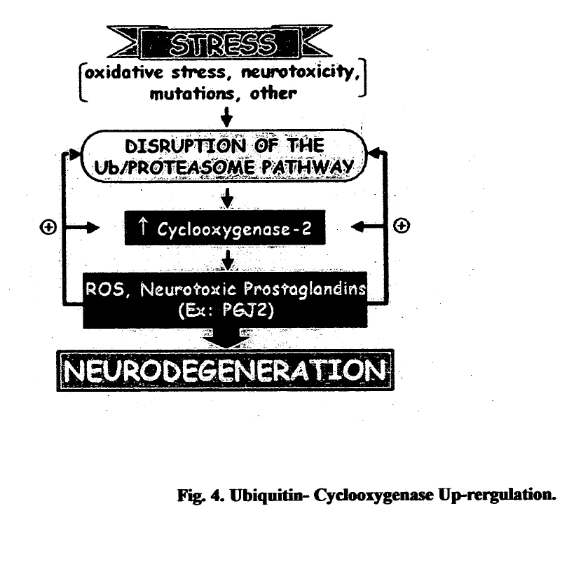

FIG. 5. Ubiquitin Lipase Inhibitors.

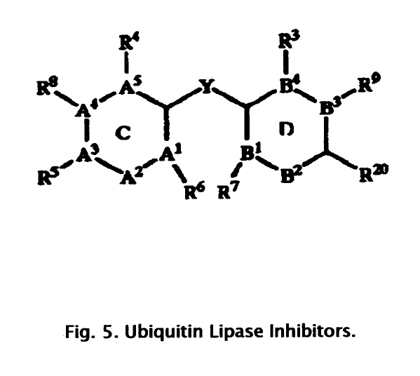

FIG. 6. Chemical backbone of Hydroxyaryl Ketones.

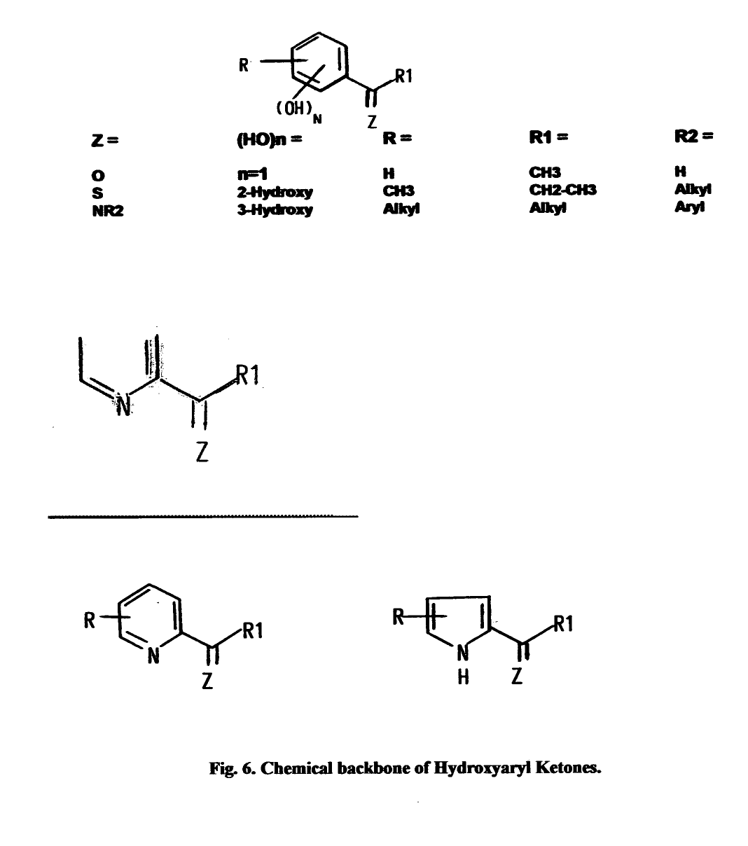

FIG. 7. Chemical Backbone of Hydroxyaryl Vinyl Ketones.

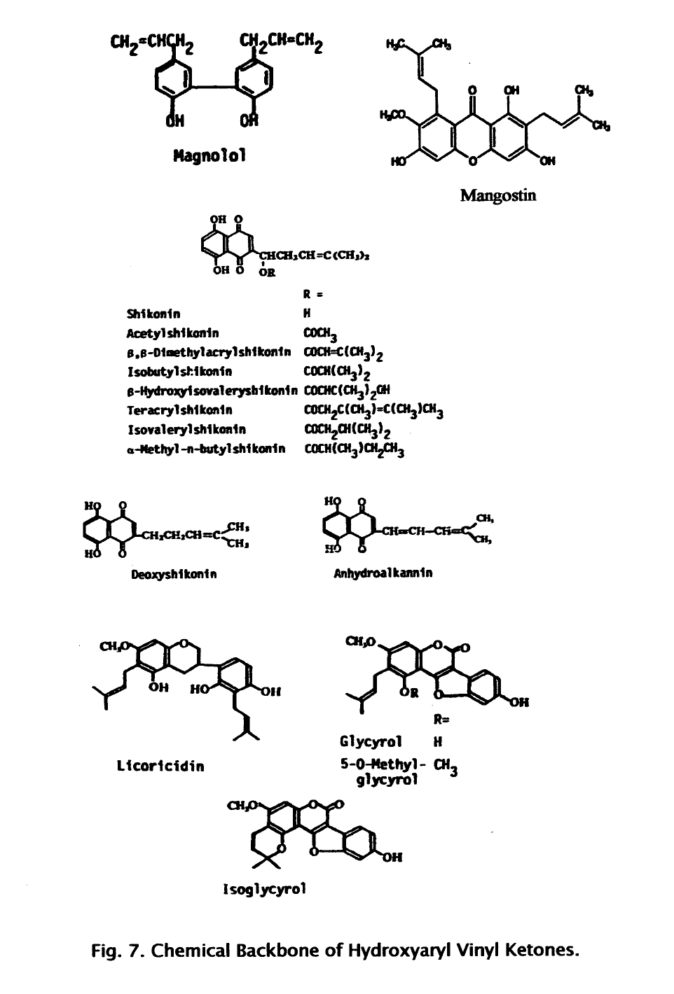

FIG. 8. Chemical Structure of Hydroxyaryl Vinyl Compounds.

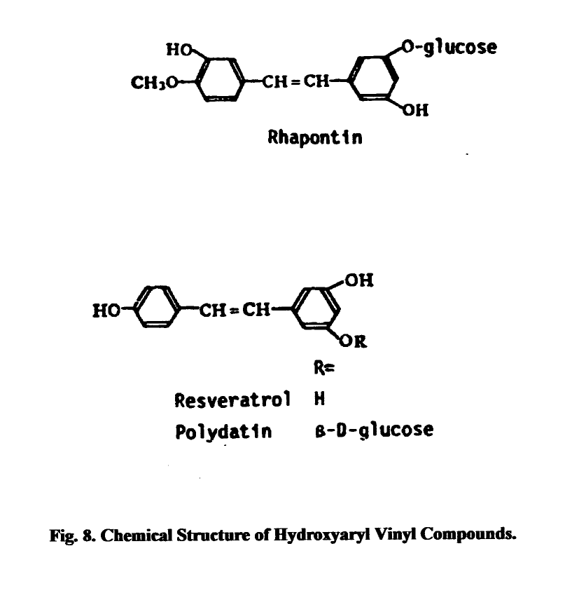

FIG. 9. Examples of Hydroxyaryl Ketones.

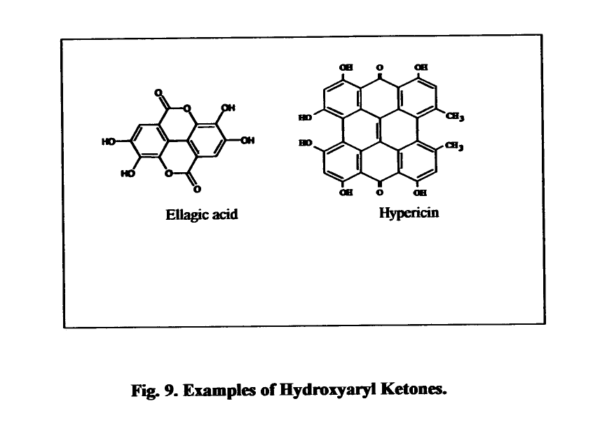

FIG. 10. Examples of Hydroxyaryl Vinyl Ketones.

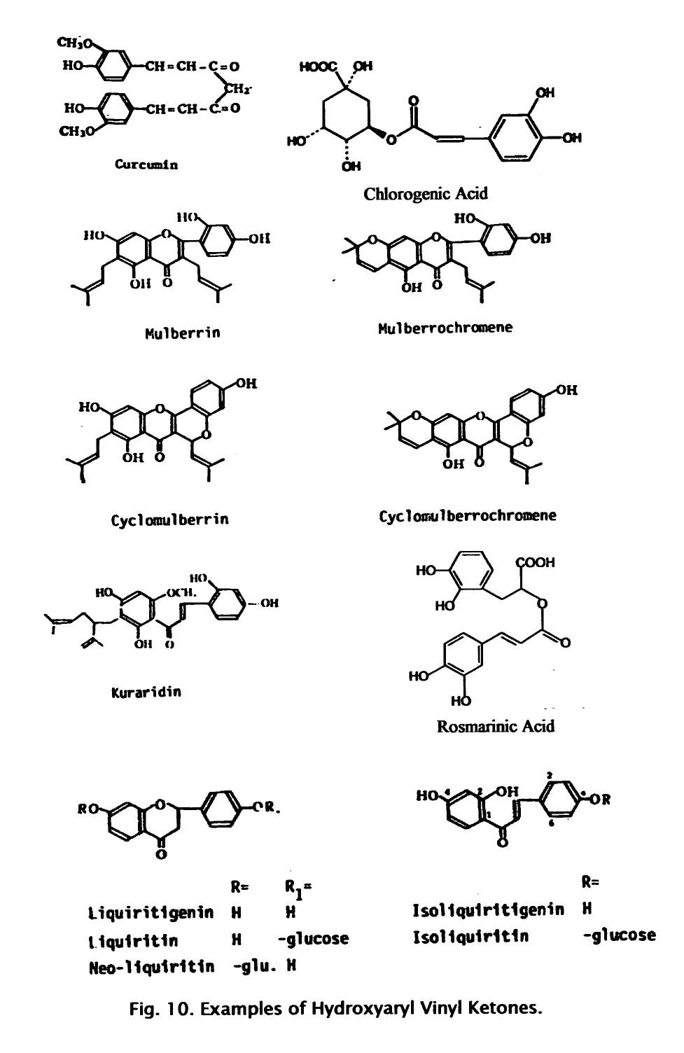

FIG. 11. Examples of Hydroxyaryl Ketones.

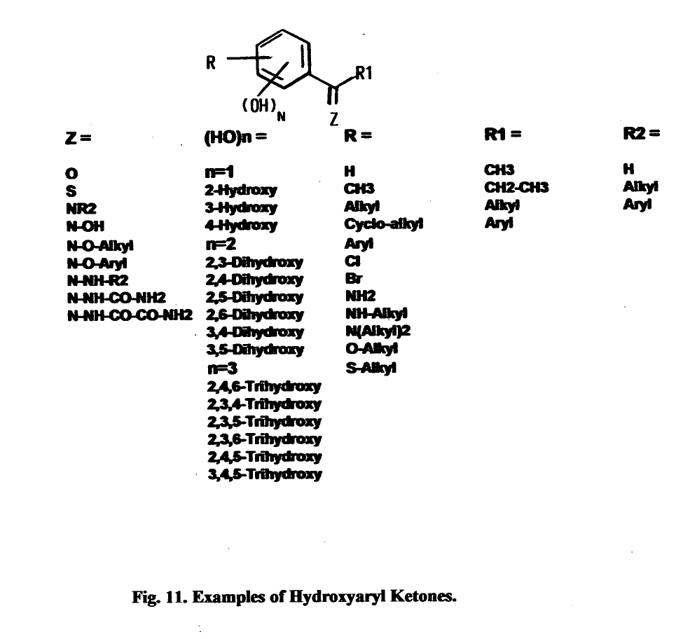

What is claimed is:

1. A molecular chaperone to attenuate the accumulation of ubiquitinylated proteins in the intracellular matrix of mammals in a cosmetically or pharmaceutically acceptable delivery system or carrier base composition.

2. A composition according to claim 1, wherein such molecular chaperone is useful for the reduction of inflammation in mammals.

3. A composition according to claim 1, wherein such molecular chaperone is useful for the treatment of skin aging, ulcer, wound healing, and enzyme malfunction related ailments in mammals.

4. A composition according to claim 1, wherein molecular chaperone compound comprising; (i) At least one hydroxyaryl, polyhydroxyaryl, or N-heterocyclic compound that contains an alkyl carbon side chain with a —C═C— group directly attached to aromatic ring.

5. A composition according to claim 1, wherein molecular chaperone compound comprising; (i) At least one hydroxyaryl, polyhydroxyaryl, or N-heterocyclic compound that contains an alkyl carbon side chain with a —C═O (ketone) group directly attached to aromatic ring.

6. A composition according to claim 1, wherein molecular chaperone compound comprising; (i) At least one hydroxyaryl, polyhydroxyaryl, or N-heterocyclic compound that contains an alkyl carbon side chain with a —CH(R)—C═C— group attached to aromatic ring via —CH(R)— moiety.

7. A composition according to claim 1, wherein molecular chaperone compound comprising; (i) At least one hydroxyaryl, polyhydroxyaryl, or N-heterocyclic compound that contains an alkyl carbon side chain with a —C(═O)—C═C— group attached to aromatic ring.

8. A composition according to claim 1, wherein molecular chaperone compound comprising; (i) At least one hydroxyaryl, polyhydroxyaryl, or N-heterocyclic compound that contains an alkyl carbon side chain with a —C═C— group as part of another aromatic ring.

9. A composition according to claim 1, wherein a cosmetically acceptable delivery system or a carrier base is selected in the form of a lotion, cream, gel, spray, thin liquid, body splash, mask, serum, solid cosmetic stick, lip balm, shampoo, liquid soap, bar soap, bath oil, paste, salve, powder, collodion, impregnated patch, impregnated strip, skin surface implant, diaper, anhydrous system, pill, syrup, tablet, capsule, and combinations thereof.

10. A composition according to claim 1, wherein such molecular chaperone is included from 0.0001% to 50.0% by weight of total composition.

11. A composition according to claim 1, wherein such molecular chaperones, when used in combination, are included from 0.0001% to 50.0% by weight of total composition.

12. A composition according to claim 4, wherein molecular chaperone compound is selected from Resveratrol, Polydatin, Rhapontin, and combinations thereof.

13. A composition according to claim 5, wherein molecular chaperone compound is selected from hydroxy or polyhydroxy acetophenones, or hydroxy or Polyhydroxy propiophenones, and their variously substituted derivatives.

14. A composition according to claim 5, wherein molecular chaperone compound is selected from Phloridzin, Ellagic acid, or combinations thereof.

15. A composition according to claim 6, wherein molecular chaperone compound is selected from Honokiol, Magnolol, Licoricidin, Glycyrol, Mulberrin, cyclo-mulberrin, and combinations thereof.

16. A composition according to claim 7, wherein molecular chaperone compound is selected from Rosmarinic acid, Curcumin, Curcuminoids, Shikonin, Hypericin, Ferulic acid, Ginkgetin, Morin, Kuraridin, Baicalin, Isoliquiritin, Rutin, iso-Pimpinellin, Xanthotoxol, Osthol, Columbianetin, Cinidiatin, Curcolone, Hyperin, and combinations thereof.

17. A composition according to claim 9, wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

18. A composition according to claim 13, wherein hydroxy or polyhydroxy acetophenone compound is selected from Resacetophenone, Quinacetophenone, Paeonol, 2-hydroxyacetophenone, 3-hydroxyacetophenone, 4-hydroxyacetophenone, 2,3-dihydroxyacetophenone, 2,4-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, 2-Acetyl resorcinol, 4-Acetyl resorcinol, 3,4-Dihydroxyacetophenone, acetyl quinol, 1-(3-Hydroxy-4-methoxy-5-methylphenyl) ethanone, 1-(3-hydroxy-4-methoxyphenyl) ethanone, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, and combinations thereof.
